(19) United States
(12) Patent Application Publication
(10) Pub. No.: US 2008/0269188 A1
(43) Pub. Date: Oct. 30, 2008

(54) Oatp-C Gene C463a Polymorphism Underlies Variable Response to Statin Therapy

(75) Inventors: John Martin Chapman, Saint Maur (FR); Philippe Giral, Paris (FR); Alain Carrie, Charenton Le Pont (FR); Sylvie Dejager, Paris (FR); Eric Bruckert, Parsi (FR)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: INSERM, Paris (FR)

(21) Appl. No.: 11/658,104

(22) PCT Filed: Jul. 20, 2005
(86) PCT No.: PCT/IB05/02626

§ 371 Date: Jun. 24, 2008

(51) Int. Cl. C12Q 1/68; A61K 31/40; A61K 31/403; A61K 31/35; A61K 31/435; A61K 31/74; A61K 31/397; A61P 9/00

(52) U.S. Cl. 514/210.02; 435/6; 514/423; 514/419; 514/460; 514/277; 424/78.35; 424/78.08

(57) ABSTRACT

The present invention relates to a method for determining variable response to statin therapy in patients afflicted with or susceptible to develop cardiovascular diseases, hypercholesterolemia, Diabetes and metabolic disorders involving high baseline plasma lipid level such as high LDL-C level, comprising detecting the presence or absence of the Pro 155T hr (C463A) variant in the Organic Anion Transporting Polypeptide-C(OATP-C) gene, wherein the presence of said variant is indicative of superior response to statin therapy. It also concerns tailored treatment of different populations of patients according to the Pro155Thr (C463A) variant genotype.

The present invention relates to a method for prognosis of patient responsiveness to treatment with statin comprising detecting the presence or absence of the Pro155Thr (C463A) variant in the Organic Anion Transporting Polypeptide-C(OATP-C) gene. It also relates to improved management of risk reduction treatments in coronary artery diseases, metabolic diseases (hypercholesterolemia, atherogenic dyslipidemias, type 2 diabetes, metabolic syndrome), stroke, peripheral vascular disease, the dyslipidemia associated with renal and neurodegenerative diseases and atherosclerosis with or without low plasma HDL-C levels.

### BACKGROUND OF THE INVENTION

Since their clinical introduction in 1987, the 3-hydroxy-3-methylglutaryl coenzyme A (HMG-CoA) reductase inhibitors (statins) have proven to be highly efficacious in reducing circulating concentration of atherogenic low-density lipoprotein cholesterol (LDL-C). Several landmark, randomized, controlled trials have demonstrated that statins reduce cardiovascular morbi-mortality both in primary and secondary prevention.¹⁻⁷ The doses used in these trials reduced plasma LDL-C levels by up to 35 percent and were associated with risk reductions in coronary artery disease of up to 37%. Recently, Cannon and associates demonstrated that aggressive statin therapy involving reduction of LDL-C levels by 51% induced a proportional reduction in major clinical outcomes.⁸

However, such reductions represent average effects in a given population. Indeed data from these large-scale clinical trials have demonstrated significant inter-individual variability in the degree of LDL-C reduction. Furthermore, little is known of the molecular basis which underlies inter-individual variability in statin response.

Numerous studies have focused on the identification of genetic determinants that may play a role in inter-individual van ability in lipid-lowering response to statins.^{9,10} Genetic polymorphisms may modulate drug response through a spectrum of mechanisms. To date, pharmacogenetic studies of statin response have focused on genes that are implicated in disease causality and have led to identification of single nucleotide polymorphisms (SNPS) in key genes of lipid metabolism including CETP, ApoE, ApoAI, ABCA1 and ABCG51G8.⁹⁻¹³ By comparison, little is known of sequence variability in genes which may directly influence statin response through pharmacokinetic or pharmacodynamic interactions. Thus, screening of two key genes implicated in the pharmacological action of statins, ie. HMG-CoA reductase and Cyp3A4, failed to reveal significant association between SNPs and drug response.^{12,14}

Among genes which code for proteins interacting directly with statins, the organic anion transporting polypeptide-C gene (OATP-C), also known as liver specific transporter-1 (LST-1) or OATP2 and ultimately as SLCO1B1 for Solute. Carrier Organic Anion Transporter Family, member 1B1, presents as an excellent candidate gene for genetically-determined modulation.^{15,16} OATP-C, which is expressed at the baso-lateral membrane of the human hepatocyte, is responsible for the hepatocellular uptake of a spectrum of endogenous and foreign substances which include statins.¹⁷⁻²⁰ Such uptake determines both intrahepatocyte and residual circulating statin concentrations and potentially constitutes one of the rate-limiting steps in the action of this class of drug. During recent years, several SNPs have been identified in the human OATP-C gene, some of which are associated with reduced in vitro transport capacity.²¹⁻²⁴ More recently, Nishizato et al. (2003) demonstrated that a commonly occurring OATP-C allele (OATP-C*15) may be associated with altered pharmacokinetics of pravastatin, in a small Japanese cohort.²⁵ Given the liver-specific tissue distribution pattern and the capacity to transport a multiplicity of substrates, OATP-C(OATP1B1 being the name of the encoded protein in the last nomenclature) has been postulated to play a role in hepatocellular drug metabolism.¹⁵⁻²⁰ Indeed, in vitro and in vivo studies have documented the specific implication of this transporter in the hepatocellular uptake of statin^{15,17,19,22,25} These findings primarily concerned pravastatin, the most hydrophilic compound in this class. The use of this specific statin was consistent with the hypothesis that lipophilic HMG-CoA reductase inhibitors, such as atorvastatin, fluvastatin, simvastatin and cerivastatin, in order of increasing relative lipophilicity²⁸, might penetrate cell membranes by passive diffusion and consequently might not involve a specific transporter. However, cis-inhibition experiments (radio-labelled pravastatin vs lipophilic statin)^{15,17}, as well as cerivastatin-cyclosporin drug-drug interaction studies²⁹, have demonstrated that OATP-C-mediated statin transport is not restricted to hydrophilic compounds, suggesting therefore that OATP-C may be implicated in the cellular uptake of all statins.

Nevertheless, as of today, no direct and significant correlation has been identified between a given polymorphism and inter-individual variability in the response to statin treatment. For example, different polymorphisms have been described in EP 1 186 672 including Asn130Asp, Arg152Lys, Val174Ala, Asp241Asn but there are no indications as to whether or not they may be implicated in different responses to statin treatment.

### SUMMARY OF THE INVENTION

In connection with the present invention, we have identified the contribution of one non-synonymous OATP-C polymorphism to inter-individual variability in response to treatment with Fluvastatin, in a European cohort of hypercholesterolemic patients. The polymorphism responsible for statin hyperresponsiveness was identified as being Pro155Thr (C463A) existing in the OATP-C*4 and OATP-C*14 allelic variants, which are prevalent among Caucasians. We further found this polymorphism heterozygous and homozygous) in about 30% of our population.

Correlation between allelic distributions in our population with plasma lipid parameters on statin treatment has revealed, for the first time in man, that OATP-C (OATP1B1) is a key factor in the therapeutic action of statins. More particularly, the Pro155Thr is functional and contributes to significant inter-individual variability in response to Fluvastatin. Based on this discovery, we provide a new tool for assessing responsiveness to statin treatment of patients, such as hypercholesterolemic patients or patients presenting other metabolic disease. We also propose new routes of treatment for low statin responder homozygous Pro155Pro patients as well as for high responder Thr155Thr and Pro155Thr patients.

### DESCRIPTION

Therefore, in a first embodiment, the invention is aimed at an ex vivo method for determining variable statin response in patients afflicted with or susceptible to develop cardiovascular diseases such as coronary artery diseases, ischaemic heart disease and myocardial infarct, Diabetes Mellitus, atherosclerosis and/or any diseases or metabolic disorders involving high baseline plasma lipid level such as high LDL-C level, as well as in renal transplantation patients, comprising detecting the presence or absence of the Pro155Thr (C463A) variant in the Organic Anion Transporting Polypeptide-C (OATP-C) gene, wherein the presence of said variant is indicative of hyperresponsiveness to statin therapy. It encompasses improved management of risk reduction treatments in coronary artery diseases, metabolic diseases (hypercholesterolemia, atherogenic dyslipidemias, type 2 diabetes, metabolic syndrome), stroke, peripheral vascular disease, the dyslipidemia associated with renal and neurodegenerative diseases and atherosclerosis with or without low plasma HDL-C levels; as well as in renal transplantation patients.

The expression “statin” will be understood herein as referring to any 3-hydroxy-3-methylglutaryl coenzyme A (HMG-CoA) reductase inhibitor including but not limited to atorvastatin (Lipitoro®), fluvastatin (Lescol®), lovastatin (Mevacor®, Altocor®), pravastatin (Pravachol®, Selektine®), rosuvastatin (Crestor®), simvastatin (Zocor®) pitavastatin (Laboratoire Kowa) as well as compounds of similar chemical formula comprising statin pharmacophore (28, incorporated herein by reference). Examples of statin formula are given below:

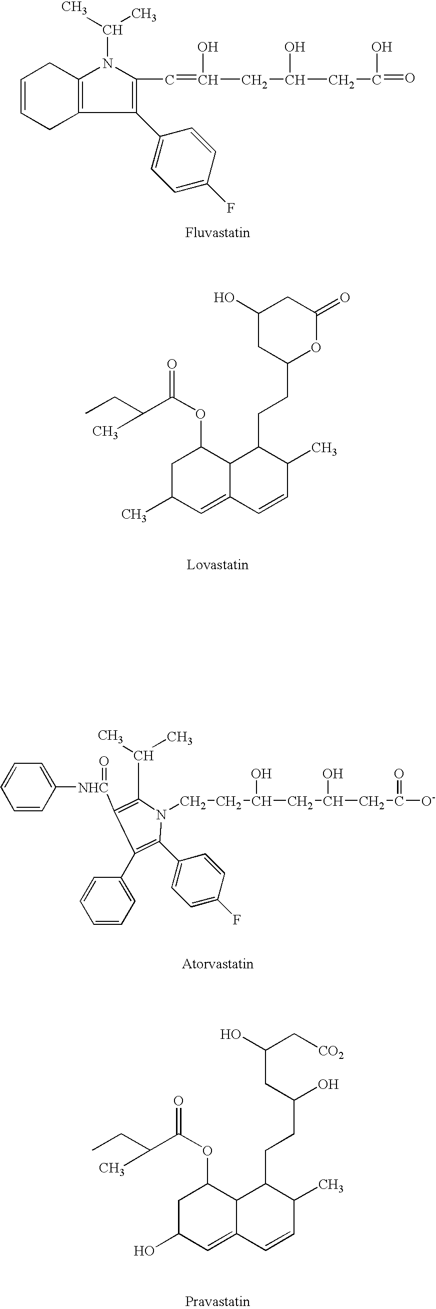

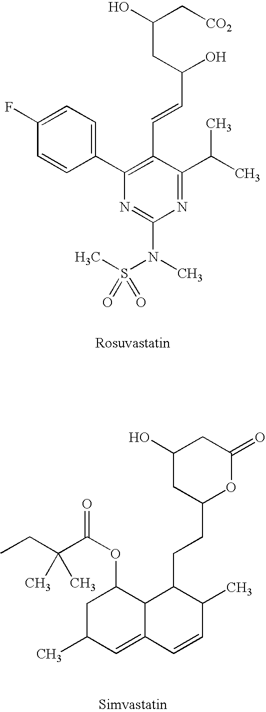

More particularly, the invention is aimed at a method as defined above for determining individual response to Fluvastatin treatment.

The term “variable response to statin therapy” means that statin treatment is more or less efficient as compared to the mean response observed in hypercholesterolemic patients. The response to a statin treatment in an individual can be assessed by very different clinical means, but especially by measuring the plasma lipid response such as Total Cholesterol, LDL-Cholesterol, HDL-Cholesterol and triglycerides plasma levels. On the basis of this variable response, it is possible to define patients sub-group of “high responder” and “low responder” to statin therapy. “High responders” correspond to the patients in whom the total cholesterol and/or LDL-C lowering response is above the average reduction observed with a defined dose of a particular statin in the population of interest. “Low responders” correspond to the patients in whom the total cholesterol and/or LDL-Clowering response is below the average reduction observed with a defined dose of a particular statin in the population of interest. For instance, in a population of hypercholesterolemic patients treated with a 80 mg daily dose of Fluvastatin XL the observed average reduction of total cholesterol and LDL-C were 25.4% and 33.1% respectively. In this population “high responders” correspond, to those with total cholesterol reduction >25.4% and/or LDL-C reduction >33.1%; whereas “low responders” correspond to those with total cholesterol reduction <25.4% and/or LDL-C reduction <33.1%.

More generically, a “high responder” patient to statin therapy is defined as displaying a reduction of total cholesterol above 25%, 30%, 35%, or even 40%, and/or a reduction of LDL-C level above 33%, 35%, or 40% after 2 months statin treatment. On the opposite, a “low responder” patient to statin therapy is defined as displaying a reduction of total cholesterol below 25%, 22%, 20%, or even 15%, and/or a reduction of LDL-C level below 33%, 30%, 25% or even 20% after 2 months statin treatment. In frame with the invention, we have found that both groups can be divided into:
homozygous Pro/Pro155 genotyped patients (low responders=70% population) and
homozygous Thr/Thr155 and heterozygous Pro/Tbr155 genotyped patients (high responders 30% population); especially in the hypercholesterolemic patients.

As used herein, the term “Organic Anion Transporting Polypeptide-C(OATP-C) gene” is used indifferently to designate the OATP-C gene (ultimately named SLCO1B1) or the encoded protein (named OAT1B1 in the last nomenclature) throughout the text. The term refer to the OATP-C of any species, especially human, but also other mammals or vertebrates to which the method of the invention can apply. The human OATP-C sequence is available under EMBL accession numbers AB026257 (OATPC, 2452 bp), AF205071 (QATP2, 2830, ref 1), AJ-132573. (OATP2, 2778), and AF060500 (LST-1) incorporated herein by reference.

For example, the AB026257 sequence is Homo sapiens mRNA for organic anion transporter OATP-C, complete cds VERSION AB026257.1 GI:5006264.

SEQ ID No 1: OATP-C (coding sequence 100-2175) showing the C463A variant being numbered from the start codon and corresponding to ABO26257:
1 gtggacttgt tgcagttgct gtaggattct aaatccaggt gattgtttca aactgagcat
61 caacaacaaa aacatttgta tgatatctat atttcaatca¹gaccaaaa tcaacatttg
121 aataaaacag cagaggcaca accftcagag aataagaaaa caagatactg caatggattg
181 aagatgttct tggcagctct gtcactcagc tttattgcta agacactagg tgcaattatt
241 atgaaaagtt ccatcattca tatagaacgg agatttgaga tatcctcttc tcttgttggt
301 tttattgacg gaagctttga aattggaaat ttgcttgtga ttgtatttgt gagttacttt
361 ggatccaaac tacatagacc aaagttaatt ggaatcggtt gtttcattat gggaattgga
421 ggtgttttga ctgctttgcc acatttcttc atgggatatt acaggtattc taaagaaact
481 aatatcaatt catcagaaaa ttcaacatcg accttatcca cttgtttaat taatcaaatt
541 ttatcactca atagagcatc a(c/a)⁴⁶³ ctgagata gtgggaaaag gttgtttaaa ggaatctggg
601 tcatacatgt ggatatatgt gttcatgggt aatatgcttc gtggaatagg ggagactccc
661 atagtaccac tggggctttc ttacattgat gatttcgcta aagaaggaca ttcttctttg
721 tatttaggta tattgaatgc aatagcaatg attggtccaa tcattggctt taccctggga
781 tctctgtttt ctaaaatgta cgtggatatt ggatatgtag atctaagcac tatcaggata
841 actcctactg attctcgatg ggttggagct tggtggctta atttccttgt gtctggacta
901 ttctccatta tttcttccat accattcttt ttcttgcccc aaactccaaa taaaccacaa
961 aaagaaagaa aagcttcact gtctttgcat gtgctggaaa caaatgatga aaaggatcaa
1021 acagctaatt tgaccaatca aggaaaaaat attaccaaaa atgtgactgg ttttttccag
1081 tcttttaaaa gcatccttac taatcccctg tatgttatgt ttgtgctttt gacgttgtta
1141 caagtaagca gctatattgg tgcttttact tatgtcttca aatacgtaga gcaacagtat
1201 ggtcagcctt catctaaggc taacatctta ttgggagtca taaccatacc tatttttgca
1261 agtggaatgt ttttaggagg atatatcatt aaaaaattca aactgaacac cgttggaatt
1321 gccaaattct catgttttac tgctgtgatg tcattgtect tttacctatt atattttttc
1381 atactctgtg aaaacaaatc agttgccgga ctaaccatga cctatgatgg aaataatcca
1441 gtgacatctc atagagatgt accactttct tattgcaact cagactgcaa ttgtgatgaa
1501 agtcaatggg aaccagtctg tggaaacaat ggaataactt acattctcacc ctgtctagca:
1561 ggttgcaaat cttcaagtgg caataaaaag cctatagtgt tttacaactg cagttgtttg
1621 gaagtaactg gtctccagaa cagaaattac tcagcccatt tgggtgaatg cocaagagat
1681 gatgcttgta caaggaaatt ttactttttt gttgcaatac aagtcttgaa tttatttttc
1741 tctgcacttg gaggcacctc acatgtcatg ctgattgtta aaattgttca acctgaattg
1801 aaatcacttg cactgggttt ccactcaatg gttatacgag cactaggagg aattctagct
1861 ccaatatatt ttggggctct gattgataca acgtgtataa agtggtccac caacaactgt
1921 ggcacacgtg ggtcatgtag gacatataat tccacatcat tttcaagggt ctacttgggc
1981 ttgtcttcaa tgttaagagt etcatcactt gttttatata ttatattaat ttatgccatg
2041 aagaaaaaat atcaagagaa agatatcaat gcatcagaaa atggaagtgt catggatgaa
2101 gcaaacttag aatccttaaa taaaaataaa cattttgtcc cttctgctgg ggcagatagt
2161 gaaacacatt gttaagggga gaaaaaaagc cacttctgct tctgtgtttc caaacagcat,
2221 tgcattgatt cagtaagatg ttatttttga ggagttcctg gtcctttcac taagaatttc
2281 cacatctttt atggtggaag tataaataag cctatgaact tataataaaa caaactgtag
2341 gtagaaaaaa tgagagtact catgtacat tatagctaca, tatttgtggt taaggttaga
2401 ctatatgatc catacaaatt aaagtgagag acatggttac tgtgtaataa aa

coding for SEQ ID No 2:
MDQNQHLNKTAEAQPSENKKTRYCNGLKMFLAALSLSFIAKTLGAIIMKS
SIIHIERRFEISSSLVGFIDGSFEIGNLLVIVFVSYFGSKLHRPKLIGIG
CFIMGIGGVLTALPHFFMGYYRYSKETNINSSENSTSTLSTCLINQILSL
NRAS(P/T)155EIVGKGCLKESGSYMWIYVFMGNMLRGIGETPIVPLGL
SYIDDFAKEGHSSLYLGILNAIAMIGPIIGFTLGSLFSKMYVDIGYVDLS
TIRITPTDSRWVGAWWLNFLVSGLFSIISSIPFFFLPQTPNKPQKERKAS
LSLHVLETNDEKDQTANLTNQGKNITKNVTGFFQSFKSILTNPLYVMFVL
LTLLQVSSYIGAFTYVFKYVEQQYGQPSSKANILLGVITIPIFASGMFLG
GYIIKKFKLNTVGIAKFSCFTAVMSLSFYLLYFFILCENKSVAGLTMTYD
GNNPVTSHRDVPLSYCNSDCNCDESQWEPVCGNNGITYISPCLAGCKSSS
GNKKPIVFYNCSCLEVTGLQNRNYSAHLGECPRDDACTRKFYFFVAIQVL
NLFFSALGGTSHVMLIVKIVQPELKSLALGFHSMVIRALGGILAPIYFGA
LIDTTCIKWSTNNCGTRGSCRTYNSTSFSRVYLGLSSMLRVSSLVLYIIL
IYAMKKKYQEKDINASENGSVMDEANLESLNKNKHFVPSAGADSETHC

### Nucleic Acid Assays:

According to a first embodiment, said C463A variant may be detected by analyzing a OATP-C nucleic acid molecule. In the context of the invention, OATP-C nucleic acid molecules include mRNA, genomic DNA and cDNA derived from mRNA. DNA or RNA can be single stranded or double stranded. These may be utilized for detection by amplification and/or hybridization with a probe, for instance.

Thus the invention provides an ex vivo method for determining statin responsiveness in patients afflicted with or susceptible to develop cadiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level; as well as in renal transplantation patients, comprising:
(a) obtaining a nucleic acid sample from the patient
(b) detecting the presence or absence of the C463A variant of OATP-C gene, in said acid nucleic sample
   wherein the presence of said variant is indicative of hyperresponsiveness to statin therapy.

The nucleic acid sample may be obtained from any cell source or tissue biopsy. Non-limiting examples of cell sources available include without limitation blood cells, buccal cells, epithelial cells, fibroblasts, or any cells present in a tissue obtained by biopsy. Cells may also be obtained from body fluids, such as blood, plasma, serum, lymph, etc. DNA may be extracted using any methods known in the art, such as described in Sambrook et al., 1989. RNA may also be isolated, for instance from tissue biopsy, using standard methods well known to the one skilled in the art such as guanidium thiocyanate-phenol-chloroform extraction.

The C463A variant of QATP-C gene may be detected in a RNA or DNA sample, preferably after amplification. For instance, the isolated RNA may be subjected to coupled reverse transcription and amplification, such as reverse transcription and amplification by polymerase chain reaction (RT-PCR), using specific oligonucleotide primers that are specific for a mutated site or that enable amplification of a region containing the variant site.

As used herein, the term “oligonucleotide” refers to a nucleic acid, generally of at least 10, preferably 15, and more preferably at least 20 nucleotides, preferably no more than 100 nucleotides, and which is hybridisable to a OATP-C genomic DNA, cDNA or 20 mRNA. Oligonucleotides can be labelled according to any technique known in the art, such as radiolabels, fluorescent labels, enzymatic label . . . . A labelled oligonucleotide may be used as a probe to detect the presence of C463A variant of OATP-C gene.

As used herein, a primer is an oligonucleotide typically extended by polymerase or litigation following hybridization to the target but a probe typically is not. A hybridised oligonucleotide may function as a probe if it used to detect a target sequence.

Therefore, useful probes or primers are those which specifically hybridize to OATP-C gene in the region of the nucleotide at position 463.

Specific probes can be preferably selected from any sequence from 10 to 35 nucleotide long surrounding and comprising the nucleotide at position 463, for example a 15 to 20 nucleotide long fragment of taatcaaatt ttatcactca atagagcatc a(c/a)⁴⁶³ctgagata gtgggaaaag gttgtttaaa (SEQ ID No 3) and comprising the nucleotide c or a at position 463. Probes may be labelled with same or different fluorescent labels to allow detection.

In a preferred embodiment, the following primers and probes can be used for the detection of the C463A (Pro155Thr) variant:

TABLE 1
Sequences of PCR Primers and MGB Probes

| | |
|---|---|
| Forward primer | 5′ AATTCAACATCGACCTTATCCACTTGT3′ |
| SEQ ID No 4 | |
| Reverse primer | 5′ ACTGTCAATATTAATTCTTACCTTTTCCCACTATC 3′ |
| SEQ ID No 5 | |
| MGB probe wildtype | 5′ VIC-CTCAATAGAGCATCACCTG-NFQ-MGB 3′ |
| SEQ ID No 6 | |
| MGB probe mutant | 5′ FAM-CAATAGAGCATCAACTG-NFQ-MGB 3′ |
| SEQ ID No 7 | |

VIC and FAM code for the reporter fluorophores, NFQ corresponds to a non-fluorescent quencher and MGB represents the minor groove binding group. Underlined nucleotides represent the location of the polymorphism.

If the method depicted above, nucleic acid may be amplified by PCR before the detection of allelic variation.

Actually other numerous strategies for genotype analysis can be used. Methods for the detection of allelic variation are described in standard textbooks, for example Molecular Cloning—A Laboratory Manual” Second Edition, Sambrook, Fritsch and Maniatis (Cold Spring, Harbor Laboratory, 1989) and Laboratory Protocols for Mutation Detection, Ed. by U. Landegren, Oxford University Press, 1996 and PCR, 2 Edition by Newton & Graham, BIOS Scientific Publishers Limited, 1997. For example, it is possible to combine an amplification step followed by a discriminative detection step. Different suitable techniques are listed in EP 1 186 672 such as DNA sequencing, sequencing by hybridization, SSCP, DGGE, TGGE, heteroduplex analysis, CMC, enzymatic mismatch cleavage, hybridization based solid phase hybridization, oligonucleotide arrays (DNA Chips), solution phase hybridization Taqman™ (U.S. Pat. No. 5,210,015 and U.S. Pat. No. 5,487,972), as well as RFLP. Detection may be performed using several possible alternative methods: FRET, fluorescence quenching, fluorescence polarisation, chemiluminescence, electrochemiluminescence, radioactivity, and colormetric.

The method of the invention may or may not include the step consisting of extracting nucleic acid from the sample as well as obtaining the sample. The sample can be blood or other body fluid or tissue obtained from an individual.

After nucleic acid extraction and purification step, PCR amplification using the above mentioned primers can be performed to improve signal detection.

Therefore, the method of the invention encompasses the step of amplification with said primers followed by the hybridization with at least one probe, more preferably two probes, specifically designed to hybridize under stringent conditions to the above sequences and the detection of the signal produce by the labels of said probes.

### Protein Assays:

According to a second embodiment said variant may be detected in MYH11 protein.

Thus the invention provides an ex vivo method for determining statin responsiveness in patients afflicted with or susceptible to develop cadiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level, comprising:
(a) obtaining sample from the patient
(b) detecting the presence or absence of the Pro155Thr variant of OATP-C protein,
   wherein the presence of said Variant is indicative of variable response to statin therapy.

Said variant may be detected according to any appropriate method known in the art. In particular a sample, obtained from the patient may be contacted with antibodies specific of the Pro155Thr variant form of OATP-C protein, i.e. antibodies that are capable of distinguishing between the Pro155Thr variant form of OATP-C protein and the wild-type protein (or any other protein).

The antibodies of the present invention may be monoclonal or polyclonal antibodies, single chain or double chain, chimeric antibodies; humanized antibodies, or portions of an immunoglobulin molecule, including those portions known in the art as antigen binding fragments Fab, Fab′, F(ab′)2 and F(v). They can also be immunoconjugated, e.g. with a toxin, or labelled antibodies.

Monoclonal antibodies are preferred rather than polyclonal antibodies because of their high specificity.

Procedures for obtaining “polyclonal antibodies” are also well known. Typically, such antibodies can be raised by administering the Pro155Thr variant form of OATP-C protein subcutaneously to New Zealand white rabbits which have first been bled to obtain pre-immune serum. The antigens can be injected at a total volume of 100 μl per site at six different sites. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody.

A “monoclonal antibody” refers to an antibody molecule which is capable of distinguishing only one epitope of an antigen. Laboratory methods for preparing monoclonal antibodies are well known in the art. Monoclonal antibodies (mAbs) may be prepared by immunizing a mammal, e.g. a mouse, rat, human and the like mammals, with a purified Pro155Thr variant form of OATP-C protein. The antibody-producing cells in the immunized mammal are isolated and fused with myeloma or heteromyeloma cells to produce hybrid cells (hybridoma). The hybridoma cells producing the monoclonal antibodies are utilized as a source of the desired monoclonal antibody. Antibody generation techniques not involving immunisation are also contemplated such as for example using phage display technology to examine naive libraries (from non-immunised animals);

Antibodies raised against the Pro155Thr variant form of OATP-C protein may be cross reactive with wild-type OATP-C protein. Accordingly a selection of antibodies specific for the Pro155Thr variant form of OATP-C protein is required, by using for instance an affinity chromatography against wild-type OATP-C protein.

Alternatively, binding agents other than antibodies may be used for the purpose of the invention. These may be for instance aptamers, which are a class of molecule that represents an alternative to antibodies in term of molecular recognition. Aptamers are oligonucleotide or oligopeptide sequences with the capacity to recognize virtually any class of target molecules with high affinity and specificity.

### Kits:

In another embodiment, the invention relates to a kit for determining statin variable response in patients afflicted with or susceptible to develop cardiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level.

According to one aspect of the invention, the kit can comprise primers and probes as defined above for detecting the presence or absence of the C463A variant in the Organic Anion Transporting Polypeptide-C(OATP-C): gene. This kit may also comprises thermoresistant polymerase for PCR amplification, one or several solutions for amplification and hybridization step, as well as any reagents allowing the detection of labels as the case may be.

But to another aspect of the invention, the kit can comprise antibodies as defined above.

The kits according to the invention can further comprise any suitable reagents for hybridization or immunological reaction such as solid-phase support.

### Therapeutic Methods:

In another embodiment, the invention concerns the fine tuning (optimized) of treatment and prevention of patients according to their genotype at position 155 of OATP-C protein.

In this regard, the invention is directed to a method for treating and/or preventing or delaying the onset of cardiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level comprising administering a decreased or increased daily dose of statin in homozygous Pro/Pro155 genotyped patients (low responders) in the Organic Anion Transporting Polypeptide-C(OATP-C) gene. Such increase or decrease may be in the range of 10 to 100%, for example from 25 to 50%, compared to the equipotent doses as shown below.

| | | Tablet sizes | Initial dose | Equipotent dose¹⁹ |
|---|---|---|---|---|
| Generic Name | Trade Name | (mg) | (mg) | (mg) |
| Atorvastatin | Lipitor | 10, 20, 40, 80 | 10, 20, 40 | 10 |
| Fluvastatin | Lescol | 20, 40 | 20 or 40 in | 80* |
| | | | evening | |
| Fluvastatin | Lescol XL | 80 | 80 in evening | 80* |
| extended release | | | | |
| Lovastatin | Generic | 10, 20, 40 | 20 in evening | 60* |
| Lovastatin | Mevacor | 10, 20, 40 | 20 in evening | 60* |
| Lovastatin | Altocor | 10, 20, 40, 60 | 20, 40, or 60 | 40*²¹ |
| extended release | | | at bedtime | |
| Pravastatin | Pravachol | 10, 20, 40, 80 | 40 | 60* |
| Simvastatin | Zocor | 5, 10, 20, 40, 80 | 20 (40 in | 20-30* |
| | | | diabetes) | |

Taken from Buse J., Clinical Diabetes Vol. 21, No 4, 2003

Also, the invention is directed to a method for treating and/or preventing or delaying the onset of cardiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level comprising administering a decreased or increased daily dose of statin in homozygous Thr/Thr155 and heterozygous Pro/Thr155 genotyped patients (high responders) in the Organic Anion Transporting Polypeptide-C(OATP-C) gene, such as an increase or decrease in the range of 10% to 100%, for example from 25% to 50%, 25% to 40%, 15% to 30% or 15% to 20% or 10% to 20%, such as for example 10%, 15%, 17%, 20%, 25%, 30%, compared to the equipotent doses as described above.

For example, regarding Fluvastatin, the invention relates to a method as depicted above wherein more than 80 mg/day, for example from 85 to 120 mg/day, 90 to 95 mg/day or 90 to 110 mg/day, for example 85, 90, 95, 100, 105, 110, 115, 120 mg/day are administered to the homozygous Pro/Pro155 genotyped patients.

The invention also relates to a method as depicted above wherein less than 80 mg/day, for example from 75 to 20 mg/day, 70 to 50 mg/day or 60 to 50 mg/day, for example 75, 70, 65, 60, 50, 45, or 40 mg/day are administered to the Thr/Thr155 and Pro/Thr155 genotyped patients.

Are specifically embraced herein, all integers between 75 to 40 and 85 to 120.

Frequency of administration may also be tailored for a patient according to its genotype at position 155 of OATP-C. For high responder patients (Thr/Thr155 and Pro/Thr155) and for low responder patients (Pro/Pro155), frequency may be increased or decreased for example from 10 to 100% or from 25 to 50% compared to current treatments (frequency current regimen).

In addition, the invention further provides combined tailored treatment and/or prevention of cardiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level comprising administering a statin and a PPARalpha agonist, such as a fibrate, according to the Pro155Thr variant in the Organic Anion Transporting Polypeptide-C(OATP-C) gene, especially to the population of low responder patients (Pro/Pro155 genotyped patients).

Among fibrates, we can cite Gemfibrozil (e.g. Lopid®), Fenofibrate, Bezafibrate (e.g. Bezalip®), Ciprofibrate (e.g. Modalim®).

In combination with statin drugs, fibrates cause an increased risk of rhabdomyolysis (idiosyncratic destruction of muscle tissue, leading to renal failure). Therefore, for high responder patients (Thr/Thr155 and Pro/Thr155), the invention is aimed at a method at defined above wherein lower doses of statin are administered combined with fibrate or lower fibrate doses are administered combined with statin or both lower fibrate and lower statin doses are associated as a combined therapy or prevention. Lower doses will be understood herein as a decrease in the range of defined above compared to current treatments.

Other combined therapy and prevention are encompassed herein for high responder patients (Thr/Thr155 and Pro/Thr155 in the Organic Anion Transporting Polypeptide-C(OATP-C) gene) and for low responder patients (Pro/Pro155):
Statin+nicotinic acid (Niacin) or derivatives (i.e Niaspan®) or other nicotinic acid receptor agonists
Statin+bile binding Resin (i.e cholestyramine, Questran®; Colesevelam, Colestipol, Welchol)
Statin+CETP inhibitors (i.e Torcetrapib®)
Statin+cholesterol adsorption inhibitors (ex Ezitimibe, Ezetrol®)
as well as any combinaison thereof (i.e statin+niacin+resin).

The invention is also generally directed to the use of statin in combination or not with fibrate, nicotinic acid, bile binding Resin, CETP inhibitors, and/or cholesterol absorption inhibitors, for the manufacture of a medicament tailored for either the Thr/Thr155 and Pro/Thr155 genotyped patients in the Organic Anion Transporting Polypeptide-C(OATP-C) gene) or for statin low responder patients (Pro/Pro155).

For example, another object of the invention is the use of Fluvastatin for preparing a medicament suitable for administration of 80 mg/day to 160 mg/day or 120 to 160 mg/day to the homozygous Pro/Pro155 genotyped patients in the Organic Anion Transporting Polypeptide-C(OATP-C) gene for treating and/or preventing or delaying the onset of cardiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level.

It is also aimed at the use of Fluvastatin for preparing a medicament suitable for administration of 20 to 40 mg/day to the Thr/Thr155 and Pro/Thr155 genotyped patients in the Organic Anion Transporting Polypeptide-C(OATP-C) gene for treating and/or preventing or delaying the onset of cardiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level.

### EXAMPLE 1

### Identification of the Pro155Thr OATP-C Gene Polymorphism Responsible for Variable Response to Statin Therapy (FAME Study)

### Methods

### Patients

The characteristics of the study subjects have been previously reported.²⁶ Patients were enrolled in a large scale, randomized, double-blind, placebo-controlled multicenter study conducted in France, Italy, Spain, Belgium and Israel. Initially, the aim of this study was to investigate the efficacy and safety of extended-release (XL) Fluvastatin 80 mg once daily for up to one year in elderly patients with primary hypercholesterolemia. The study was approved by the relevant Ethic Committees, and informed consent Was given by all subjects before entering the study. Men or women, aged 70-85 years, with primary hypercholesterolemia (total cholesterol ≧251 mg/dL, triglycerides ≦407 mg/dL and LDL cholesterol ≧159 mg/dL after dietary intervention) were eligible for inclusion. The main exclusion criteria were: type I or V hyperlipoproteinemia (WHO classification) with hyperlipidemia secondary to other causes, or a total cholesterol:HDL cholesterol ratio <4-0; severely impaired renal function (creatinine clearance <30 mL/min); symptomatic congestive heart failure; history of myocardial infarction, angina pectoris or stroke; severe peripheral arterial disease (Fontaine stage III or IV); and a history of muscle disease. Patients currently taking a lipid-modulating drug were eligible after a 4-week washout period. The study comprised a screening visit at baseline (2 weeks before the study), and a double-blind treatment period that continued to the end of the study. At week 0, patients were randomized to receive fluvastatin XL 80 mg or placebo, once daily at bedtime. Medications prohibited during the study included all lipid-modulating agents other than fluvastatin XL, anticoagulants (at randomization), cyclosporin and erythromycin (given systemically and continuously). Blood was taken for DNA extraction once at baseline and for lipid analysis at week—2, and then at each visit (weeks 0, and 2, 8 and every 6 months after randomization). A total of 1229 subjects were randomized and received the study treatment (Fluvastatin XL 80 mg: n=607; placebo: n=622). OATP-C genotypes were determined in 420 subjects from the Fluvastatin arm of the study.

### Procedures

For each subject, plasma lipid response was calculated based on values obtained at baseline (week—2) and 2 months after treatment. Laboratory methods for lipid and lipoprotein measurements are described elsewhere 26; all parameters were measured at a central laboratory. DNA was extracted from white blood cells using standard protocols. Genotyping assays of SNPs were developed using the Assays-by-Design^{SM} service from Applied Biosystems (myscience.appliedbiosystem.com, Foster City, Calif.). Briefly, after submission, for each polymorphism, of the sequence containing the target variant, this development service designs, synthesizes, formulates and delivers primer and probe sets for SNP genotyping based on allelic discrimination using the 5′ nuclease assay with Taqman® probe using Minor Groove Binder (MGB) DNA oligonucleotide technology.²⁷ The Assays-by-Design^{5M} service delivers assay reagents for the genotyping of specific SNP consisting of a 40× mix of unlabeled PCR primers and Taqman® MGB probes (FARM and VIC® dye-labeled). PCR primers and probes for the detection of C463A (Pro155Thr) OATP-C polymorphisms are listed in Table 1. Each genotyping reaction was performed in a final volume of 25 μl containing 12·5 μl of Taqman® Universal PCR master mix, 0-625 μl of 40× Assay mix and 15 to 25 ng of genomic DNA diluted in 11·875 μl H₂O. The reactions were submitted to thermal cycling (95° C. for 10 min and 40 cycles with 92° C. for 15 s and 60° C. for 0.1 min) in an ordinary cycler (GeneAmp PCR system 9700, Applied Biosystems). Endpoint fluorescence (FAM™, VIC® or both), corresponding to cleavage of the allele-specific probe (allelic discrimination) was measured using an ABI PRISM 7000 Sequence Detection System (Applied Biosystems, Foster City, Calif.).

### Statistical Analysis

Mean ±SD (standard deviation) are given for all continuous variables and absolute numbers and percentages for sex and genotypes. A logarithmic transformation was applied to plasma triglyceride values before statistical analysis. Differences between lipid parameters at baseline and at treatment were tested using paired t test. To evaluate the effects of allelic distribution on drug response, several stages were compared. First, 1-way analysis of variance was used to evaluate potential differences between the three genotypes of each polymorphism. When the ANOVA showed a significant global difference (p<0.05), we tested the differences between genotypes two by two by means of the Tukey Kramer HSD test, and equally performed a test for linear trend between column means and column number for the three genotypes. Second, we used a stepwise model for multivariate linear regression in order to estimate the relative contribution of each studied variable to the decrease of LDL-cholesterol level. In this multivariate analysis, subjects who were homozygous for the wild-type allele were compared with those carrying one or more variant allele (dominant model), or subjects carrying one or more wild-type allele were compared with those who were homozygous for the variant allele (recessive model). Statistically significant variables selected by the forward stepwise procedure were then included in a new model and the standard least square procedures were applied. A probability value <0.05 was considered significant. We used the JMN5 (SAS institute, Cary, N.C., USA) software for Windows for all statistical analyses.

### Results

In our study group (98 males, 322 females), the mean (±SD) age was 75·5±3·8 years and BMI 26·5±4.1 kg/m²; 56% (n=236) of the patients presented high blood pressure, 6% (n=25) presented diabetes mellitus, 4% (n=18) were smokers and 25% (n=104) had a family history of cardiovascular disease. Plasma lipid parameters (means ±SD), before and after treatment with Fluvastatin XL in these 420 hypercholesterolemic subjects, are given in Table 2.

TABLE 2
Plasma lipid parameters in the study group (420 subjects),
before and after treatment with Fluvastatin XL (80 mg).

| | Baseline | Treatment | | |
|---|---|---|---|---|
| | (mg/dL) | (mg/dL) | Change % | p Values* |
| TC | 284 ± 33 | 210 ± 37 | −25.4 ± 12.7 | <0.0001 |
| LDL-C | 202 ± 29 | 134 ± 33 | −33.1 ± 16.3 | <0.0001 |
| HDL-C | 54 ± 10 | 53 ± 11 | −0.97 ± 14.4 | 0.03 |
| TC/HDL-C | 5.5 ± 1.1 | 4.1 ± 1.0 | −23.8 ± 14.1 | <0.0001 |
| TG | 145 ± 55 | 120 ± 45 | −13.5 ± 26.3 | <0.0001 |

Values are mean ± SD (standard deviation)
*comparison by paired t test between baseline and treatment lipid values.

As expected, we observed a significant 33% mean reduction in LDL-C level (p<0.0001) after 2 months statin treatment in the study group. The genotype distributions for each polymorphism are listed in Tables 3; all were consistent with the Hardy-Weinberg equilibrium. In our population, the Pro155Thr (C463A) and Val174Ala (T521C) variants were found at the same frequency, 17% and 14% respectively, as previously described in Caucasians.^{21,22}

TABLE 2BIS
Placebo arm of the FAME study.
SNP_5304 Pro155Thr

| | AA | AC | CC | p Value |
|---|---|---|---|---|
| Placebo n = | 13 | 83 | 208 | |
| LDL_Vis2 | 200 +/− 45 | 195 +/− 35 | 199 +/− 34 | 0.5751 |
| Vis2_% change | −2.22 +/− 18.13 | −2.75 +/− 14.89 | −2.15 +/− 12.62 | 0.9424 |
| LDL_base | 206 +/− 30 | 201 +/− 32 | 205 +/− 30 | 0.6835 |
| TCHO_Vis2 | 283 +/− 46 | 278 +/− 40 | 280 +/− 41 | 0.8517 |
| Vis2_% change | −1.04 +/− 13.18 | −2.52 +/− 11.34 | −1.96 +/− 10.50 | 0.8705 |
| TCHO_base | 287 +/− 32 | 286 +/− 38 | 287 +/− 35 | 0.9941 |
| TRI_Vis2 | 135 +/− 58 | 153 +/− 56 | 154 +/− 66 | 0.5697 |
| Vis2_% change | −8.91 +/− 32.18 | 2.84 +/− 25.38 | 4.47 +/− 28.68 | 0.2423 |
| TRI_base | 150 +/− 38 | 152 +/− 52 | 150 +/− 56 | 0.9565 |
| HDL_Vis2 | 56 +/− 14 | 53 +/− 13 | 50 +/− 11 | 0.0548 |
| Vis2_% change | 10.41 +/− 25.48 | −3.06 +/− 10.30 | −3.36 +/− 10.84 | 0.0002 |
| HDL_base | 51 +/− 11 | 55 +/− 11 | 52 +/− 10 | 0.1838 |

As depicted in Table 2bis, no significant differences between polymorphisms has been observed regarding placebos.

In the case of the Pro155Thr variant (C463A), the ANOVA procedure (Table 3) demonstrated highly significant associations between the different genotypes (CC, CA and AA) and both mean post-treatment LDL-C values (p=0-0005) and % LDL-C reduction (p=0-005). The CC (Pro/Pro) homozygous subjects (n=294, 70%) exhibited post-treatment LDL-C values (138 mg/dL) and mean % LDL-C reduction (−31·5%) which were significantly greater and lower, respectively, than those (126 mg/dL; −36·2%) of the heterozygous CA (Pro/Thr) patients (n=111; 26%) on the one hand, and of those (115 mg/dL; −41%) of the homozygous AA (Thr/Thr) subjects (n=15; 4%) on the other. Total cholesterol values (post-treatment, % reduction) were also significantly associated with C463A genotypes. Tukey Kramer HSD analysis, using two by two comparisons among Pro155Thr genotypes, confirmed the significance of the differences in absolute post-treatment values and % changes for both total and LDL-cholesterol, at least between wild-type homozygous (CC) and heterozygous (CA) individuals (Table 3). The statistical significance of the Pro155Thr variant effects was reinforced by a significant (p=0-03) linear trend for mean reduction in LDL-C (% change).

TABLE 3
Plasma lipid parameters before and after Fluvastatin XL treatment according
to C463A (Pro 155Thr) polymorphism.

| | | | | p Values | | | |
|---|---|---|---|---|---|---|---|
| | | | | | C vs | C vs | |
| | CC | CA | AA | ANOVA | CA* | A* | CA vs A* |
| N°. of Subjects | 294 (70%) | 111 (26%) | 15 (4%) | | | | |
| Baseline (mg/dL) | | | | | | | |
| TC | 285 ± 33 | 283 ± 32 | 277 ± 39 | 0.65 | — | — | — |
| LDL-C | 203 ± 29 | 200 ± 28 | 197 ± 35 | 0.50 | — | — | — |
| HDL-C | 53 ± 11 | 55 ± 10 | 52 ± 13 | 0.39 | — | — | — |
| TC/HDL- | 5.5 ± 1.2 | 5.3 ± 1.0 | 5.5 ± 1.0 | 0.18 | — | — | — |
| C | | | | | | | |
| TG | 145 ± 53 | 145 ± 58 | 141 ± 61 | 0.94 | — | — | — |
| Treatment (mg/dL) | | | | | | | |
| TC | 215 ± 37 | 204 ± 32 | 188 ± 39 | 0.001 | S | S | — |
| LDL-C | 138 ± 34 | 126 ± 29 | 115 ± 33 | 0.0005 | S | S | — |
| HDL-C | 53 ± 12 | 54 ± 10 | 50 ± 13 | 0.34 | — | — | — |
| TC/HDL- | 4.2 ± 1.1 | 3.9 ± 0.8 | 3.9 ± 1.0 | 0.003 | S | — | — |
| C | | | | | | | |
| TG | 122 ± 46 | 118 ± 45 | 114 ± 45 | 0.58 | — | — | — |
| % Change | | | | | | | |
| TC | −24.2 ± 12.8 | −27.7 ± 12.0 | −32.0 ± 12.7 | 0.006 | S | — | — |
| LDL-C | −31.5‡ ± 16.4 | −36.2‡ ± 15.5 | −41.0‡ ± 14.2 | 0.005 | S | — | — |
| HDL-C | −0.77 ± 15.0 | −1.0 ± 12.8 | −4.5 ± 15.4 | 0.60 | — | — | — |
| TC/HDL- | −22.7 ± 14.6 | −26.2 ± 13.0 | −28.4 ± 11.2 | 0.04 | — | — | — |
| C | | | | | | | |
| TG | −13.3 ± 23.5 | −13.6 ± 33.2 | −16.7 ± 21.6 | 0.90 | — | — | — |

Values are mean ± SD or n (percentage).
*two by two comparisons by Tukey Kramer HSD, S means significant with pvalue <0.05.
†values (means, population numbers) used for the test for linear trend.

A stepwise forward multiple regression analysis including all parameters (age, gender, BMI, baseline lipid values and C463A genotypes) allowed us to conclude that gender and BMI were not correlated with mean LDL-C reduction. The standard least square procedures applied to a new model including all the remaining parameters demonstrated that baseline triglycerides (log-transformed), age, C463A genotypes in a dominant model and baseline LDL-C were independent predictors of LDL-C reduction (Table 4).

TABLE 4
Results of multivariate regression analysis for reduction of LDL-C
level after Fluvastatin XL treatment.

| Variables | β (SE) | p Values |
|---|---|---|
| Age | 0.7 (0.2) | 0.0007 |
| Baseline triglycerides | −9.7 (5.1) | 0.056 |
| Baseline LDL-C | −5.5 (1.1) | <0.0001 |
| Genotypes C463A, dominant model | −3.1 (0.9) | 0.0004 |

β indicates regression coefficient,
SE means standard error.

In this multivariate analysis, the Pro155Thr genotype was the most significant factor influencing drug response after baseline LDL-C level.

### Discussion:

In the present example, we demonstrate for the first time in man that the Pro155Thr (C463A) variant in OATP-C gene is significantly and independently associated with a more efficacious LDL-lowering response to Fluvastatin treatment. In our population, this genetically-determined response confers an absolute gain of 10% (−41% vs −315%) in LDL-C reduction in the homozygous Thr/Thr subjects as compared to the homozygous wild-type Pro-Pro patients. Such biological benefit was equally observed for total plasma cholesterol levels.

Our present results in a clinical trial with lipophilic fluvastatin demonstrate that OATP-C mediates the hepatocellular uptake of all statins in man. In light of such substrate specificity, statin-mediated OATP-C-transport may involve direct interaction between specific OATP-C amino acid residues and the statin pharmacophore 28, a feature potentially shared by all statins.

Several non-synonymous polymorphisms have been reported in the OATP-C coding sequence²¹⁻²³, notably in regions linked to substrate specificity, but until the present invention, data in man on the impact of these polymorphisms on biological response and clinical outcome following statin treatment Were lacking.

In our population, and despite in vitro and pharmacokinetic data showing altered transport capacity 212225, the Val174Ala (T521C) polymorphism was not significantly associated with changes in plasma lipid parameters on fluvastatin treatment. Moreover, the moderate impact of this polymorphism on “in vivo” fluvastatin response may also be explained by substrate specific effects, as the reduced in vitro trans port activity reported for this SNP was observed with estrone sulphate and estradiol 17 β-D glucuronide.²¹

In contrast, the Pro155Thr (C463A, existing in OATP-C*4 and OATP-C*14) polymorphism was associated with a highly significant, genetically-determined modulation of fluvastatin response which involved both total and LDL-cholesterol levels. Interestingly, previous in vitro experiments using different substrates (estrone sulphate and estradiol 17 β-D glucuronide) on OATP-C*4 transfected cells did not reveal pronounced modification in OATP-C transport efficiency.^{21,22} In addition, there is a lack of pharmacokinetic data on the Pro155Thr (C463A, OATP-C*4 OATP-C*14) variant, as this SNP was not detected in the Japanese population in which the contribution of OATP-C polymorphisms to statin pharmacokinetics was performed.²⁵However, in support of the functional significance of this polymorphism, this nucleotidic transversion leads to a proline to threonine substitution, involving a marked change in amino acid with conformational consequences (loss of a proline) and potential post-translational modifications (O-glycosylation of the threonine residue). Moreover, the Pro155Thr substitution is located in extracellular loop 2 of OATP-C in which amino acid changes are presumed to affect substrate specificity.²⁴

In our study, the variant allelic frequencies of the Pro155Thr (C463A) and Val174Ala (T521C) polymorphisms (17% and 14% respectively) were consistent with those previously reported in the same ethnic group.^{21,22} In addition, genotype, distributions for these polymorphisms were in accordance with Hardy-Weinberg equilibrium and did not exhibit any association with baseline mean plasma lipid parameters. Therefore, our present results are not linked to the specific demographic features and gender of our population (mean age of 75.5 years and 77% of women).

Homozygous Thr/Thr subjects exhibited an absolute gain of 8% (−32% vs −24-2%) in total cholesterol lowering as compared to homozygous Pro/Pro, whereas heterozygous Pro/Thr patients showed an intermediate absolute increase of 3.5% (27-7% vs 24.2%) in total cholesterol reduction. The Pro155Thr variant may also confer clinical benefit as a consequence of its impact on on-treatment plasma LDL-C levels. Analysis of the results of the Scandinavian Simvastatin Survival Study (4S) 1 showed that major coronary event rates over 5 years were 18-9% in patients with on-treatment plasma LDL-C levels of 127 to 266 mg/dL, 13-3% in those with plasma LDL-C levels of 105 to 126 mg/dL, and 10-8% in those with plasma LDL-C levels of 58 to 104 mg/dL. As a function of the Pro155Thr genotype, the third of our population carrying one or more variant allele(s) (dominant model) displayed mean on-treatment plasma LDL-C levels below 126 mg/dL, instead of 138 mg/dL for 70% of our population with the wild-type allele.

In conclusion, the Pro155Thr polymorphism appears to be functionally involved in the pharmacological action of statins as it contributes significantly to inter-individual variability in statin response in one third of the population. Our findings have potentially wide-ranging implications for lipid-lowering therapy in atherogenic dyslipidemias, notably as a consequence of the integration of pharmacogenetic factors into the therapeutic strategy for optimal clinical benefit.

### EXAMPLE 2

### A Retrospective Pharmacogenetic Analysis of Polymorphisms in the OATP-C Gene in the ALERT Trial (Assessment of LEscol in Renal Transplantation)

A retrospective pharmacogenetic analysis was conducted in an attempt to replicate associations between a genetic variation in the OATP-C gene (Slc21A6) and cholesterol parameters in response to fluvastatin in the Fluvastatin/Lescol® ALERT clinical trial.

### Material and Methods

### Patients

Given the success of statins, and fluvastatin in particular, in lowering lipid levels and reducing cardiovascular disease in the general population, the Assessment of LEscol (Fluvastatin) in Renal Transplantation (ALERT) clinical trial was performed (Holdaas et al., 2003). This multicenter, randomized, double-blind placebo-controlled study followed 2012 renal transplant patients for 5-6 years. Patients were assigned to a placebo or fluvastatin (40 mg daily for two years, and 80 mg daily for the remainder of the study) group, and monitored in laboratory blood analysis every six months and annual electrocardiography.

The Alert trial was conducted in centres in the Scandinavian countries, UK, Germany, Belgium, Switzerland and Canada, with only minimal number of non-Caucasian patients. The demographic and baseline LDL-C characteristics are similar between the fluvastatin treatment and the placebo groups, as illustrated in Table 5.

TABLE 5
Demographic and baseline LDL-C characteristics

| | Placebo | | | | Fluvastatin | | | |
|---|---|---|---|---|---|---|---|---|
| | | | | LDL | | | | LDL |
| Gender | N = | BMI | Age | Baseline | N = | BMI | Age | Baseline |
| Male | 444 | 25.75 ± | 49.94 ± | 4.15 ± | 477 | 25.78 ± | 49.88 ± | 4.14 ± |
| | | 4.17 | 10.90 | 1.02 | | 4.03 | 10.90 | 0.97 |
| Female | 248 | 25.74 ± | 51.5 ± | 4.24 ± | 229 | 25.93 ± | 48.97 ± | 4.16 ± |
| | | 5.39 | 10.61 | 0.98 | | 5.16 | 10.48 | 1.03 |

### Procedures:

Blood samples from each consenting patient were collected at the individual trial sites. The genomic DNA of each patient was extracted from the blood using the PUREGENE™ DNA Isolation Kit (D-50K) and then genotyping was performed. Ultimately, 1375 ALERT samples were genotyped: 693 from the Fluvastatin group (of 1050 total patients) and 682 from the placebo group (of 1052 total patients).

The primary efficacy variables tested were: LDL cholesterol at visit 2 (6 weeks of treatment) and Change in LDL cholesterol. The change in LDL cholesterol was calculated as the difference between the week 6 value and the visit zero value, for patients for whom both numbers were available. Additional efficacy variables tested in the full set of genotypes were:
HDL cholesterol at visit 2 (6 Weeks of treatment)
Change in HDL cholesterol
Total cholesterol at visit 2 (6 weeks of treatment)
Change in total cholesterol
Triglycerides at visit 2 (6 weeks of treatment)
Change in triglycerides.

Covariates in the genotype-phenotype association analysis were:
Baseline value
Treatment center
Gender

All were drawn directly from the clinical data set.

The 6 following polymorphisms in this gene have been genotyped:

TABLE 6
Genotyped polymorphisms

| CPG ID# | Gene | Reference | Description |
|---|---|---|---|
| 4817 | OATP-C | rs2291075 | Synonymous (F199F) |
| 4818 | OATP-C | rs2306283 | Missense (T388C or N130D) |
| 4876 | OATP-C | hCV1901734 | 3′ UTR |
| 4877 | OATP-C | hCV1901779 | Intron |
| 5304 | OATP-C | rs11045819 | Missense (C463A or P155T) |
| 5305 | OATP-C | rs4149056 | Missense (T521C or V174A) |

As shown in the results only polymorphism at position 155 (SNP_{—}5304 P155T) is associated with modulation of statin treatment response. The other five SNPs tested did not show any implication in statin response in patients.

SNP assays were designed using information from the public dbSNP database, the proprietary Celera/ABI database or from FAME study (example 1). The resulting probe sets for the genotyping assay were generated for ABI's Assays-by-Design® platform (Livak et al. 1995). Genotyping was performed on 10 ng of genomic DNA according to the manufacturer's instructions. The results were stored in the Clinical Pharmacogenetics database after quality checking.

### Statistical Analysis:

Initial analysis of allele frequencies and conformance to Hardy-Weinberg equilibrium was performed. Genotype-phenotype association studies and related analyses were performed in SAS (Cary, N.C.) using a scripted workflow designed for this project.

Association tests used categorical genotypes as the independent variable, with no assumption about dominance, and the various efficacy variables as dependent variables. Tests of continuous dependent variables used an ANCOVA analysis. No adjustment was made for multiple testing effect.

The relevant association test for each phenotype was first performed in the Fluvastatin treated patient set. SNP-phenotype associations with a threshold of p<0.05 were then tested separately in the placebo set to determine Fluvastatin-related pharmacogenetic effect.

### Results

Significant associations were seen between A463C (Pro155Thr) in OATP-C and LDL cholesterol and total cholesterol reduction in response to fluvastatin treatment, as reported before in example 1 (FAME study).

Indeed, we replicated the Pro155Thr association with LDL-C and TC values at 6 week time point in the fluvastatin treated arm, but not in placebos. The A463C SNP was associated with both reduction from baseline and post-treatment values after 6 weeks of fluvastatin treatment for the LDL-C and TC parameters (p=0.0008 and 0.0181), but not for HDL-C and TG parameters. Likewise, no significant association with baseline values was seen.

In the experiments of example 1, another non-synonymous polymorphism in the OATP-C gene, Val174Ala was studied, and no significant association with LDL-C reduction and post-treatment value was identified. We observed the same with regard to this SNP and 4 additional SNPs included in Table 5 in the ALERT trial.

TABLE 7
Effect of OATP-C Pro155Thr variation on lipid parameters

| | AA | AC | CC | p Value |
|---|---|---|---|---|
| fluvastatin n = | 16 | 167 | 468 | |
| LSMEAN | | | | |
| LDL_Visit 2 | 2.68 | 3.12 | 2.91 | 0.0008 |
| LDL_Diff | −1.45 | −1.02 | −1.22 | 0.0008 |
| LDL_% change | −34% | −23% | −28% | 0.0034 |
| LDL_baseline | 3.93 | 4.23 | 4.22 | 0.4925 |
| TC_Visit 2 | 5.17 | 5.39 | 5.21 | 0.0181 |
| TC_Diff | −1.27 | −1.05 | −1.23 | 0.0181 |
| TC_% change | −20% | −16% | −18% | 0.0133 |
| TC_baseline | 6.28 | 6.58 | 6.54 | 0.579 |
| TG_Visit 2 | 2.12 | 1.98 | −1.93 | 0.4468 |
| TG_Diff | −0.037 | −0.181 | −0.226 | 0.4468 |
| TG_% change | 8% | −3% | −5% | 0.1807 |
| TG_baseline | 1.78 | 2.15 | 2.05 | 0.411 |
| HDL_Visit 2 | 1.58 | 1.4 | 1.43 | 0.0888 |
| HDL_{——}Diff | 0.23 | 0.05 | 0.08 | 0.0888 |
| HDL_% change | 18% | 8% | 9% | 0.4091 |
| HDL_baseline | 1.47 | 1.38 | 1.42 | 0.612 |

### Discussion

This analysis replicated the finding from FAME study with regard to Pro155Thr variation and its significant associations with lipid variables (LDL-C and TC) in response to fluvastatin treatment. However, a difference has been observed between the two studies for the Pro/Thr heterozygotes. This is probably due to the fact that the ALERT study is very different from the study of example 1 (FAME trial) in several regards. First, the patient populations were elderly hypercholesterolemia patients in FAME, with baseline LDL-C average 200 mg/dL, whereas the patients were renal transplantation patients in ALERT, with baseline LDL-C ˜160 mg/dL.

In addition, fluvastatin drug dose was twice as much in the FAME trial (80 mg vs 40, mg). Thus, the data presented in Table 4 regarding the Pro/Thr heterozygotes show that at lower doses of fluvastatin, it is more difficult to see a difference between the groups.

Second, the age of patients in the ALERT trial ranged from 23 to 74 years, compared to 70-85 years in the FAME trial. However, when divided into age groups, there was no age effect observed. The 60-80 years age group showed the same pattern of lipid parameter distribution as the whole group.

To conclude, we can distinguished for renal transplantation patients that the Pro/Pro homozygotes are low responders compared to the Thr/Thr homozygotes.

### REFERENCES

1 Randomised trial of cholesterol lowering in 4444 patients with coronary heart disease: the Scandinavian Simvastatin Survival Study (4S). Lancet 1994; 344(8934):1383-9.
2 Sacks F M, Pfeffer M A, Moye L A, et al. The effect of pravastatin on coronary events after myocardial infarction in patients with average cholesterol levels.

Cholesterol and Recurrent Events Trial investigatots. N Engl J Med 1996; 335(14):1001-9.
3 Prevention of cardiovascular events and death with pravastatin in patients with coronary heart disease and a broad range of initial cholesterol levels. The Long-Term Intervention with Pravastatin in Ischaemic Disease (LIPID) Study Group. N Engl J Med 1998; 339(19):1349-57.
4 MRC/BHF Heart Protection Study of cholesterol lowering with simvastatin in 20,536 high-risk individuals: a randomised placebo-controlled trial. Lancet 2002; 360(9326):7-22.
5 Shepherd J, Cobbe: S M, Ford I, et al. Prevention of coronary heart disease with pravastatin in men with hypercholesterolemia West of Scotland Coronary. Prevention Study Group. N Engl J Med 1995; 333(20):1301-7.
6 Downs J R, Clearfield M, Weis S, et al. Primary prevention of acute coronary events with lovastatin in men and women with average cholesterol levels: results of AFCAPS/TexCAPS. Air Force/Texas Coronary Atherosclerosis Prevention Study. Jama 1998; 279(20):1615-22.
7 Shepherd J, Blauw G J, Murphy M M, et al. Pravastatin in elderly individuals at risk of vascular disease (PROSPER): a randomised controlled trial. Lancet 2002; 360(9346):1623-30.
8 Cannon C P, Braunwald E, McCabe C H, et al. Intensive versus moderate lipid lowering with statins after acute coronary syndromes. N Engl J Med 2004; 350(15):1495-504.
9 Maitland-van der Zee A H, Klungel O H, Stricker B H, et al. Genetic polymorphisms: importance for response to HMG-CoA reductase inhibitors. Atherosclerosis 2002; 163(2):213-22.
10 Schmitz G, Drobnik W. Pharmacogenomics and pharmacogenetics of cholesterol-lowering therapy. Clin Chem Lab Med 2003; 41(4):581-9.
11 Lahoz C, Pena R, Mostaza J M, et al. Apo A-I promoter polymorphism influences basal HDL-cholesterol and its response to pravastatin therapy. Atherosclerosis 2003; 168(2):289-95.
12 Messer C, Rounds E, Zhan P, et al. HMGCR and ABCA1 variability as predictors of statin response. Atherosclerosis Supplements 2003; 4(2):136.
13 Kajinami K, Brousseau M E, Nartsupha C, Ordovas J M, Schaefer E J. ATP binding cassette transporter G5 and G8 genotypes and plasma lipoprotein levels before and after treatment with atorvastatin. J Lipid Res 2004; 45(4):653-6.
14 Kajinami K, Brousseau M E, Ordovas J M, Schaefer E J. CYP3A4 genotypes and plasma lipoprotein levels before and after treatment with atorvastatin in primary hypercholesterolemia. Am J Cardiol 2004; 93(1): 104-7.
15 Hsiang B, Zhu Y, Wang Z, et al. A novel human hepatic organic anion transporting polypeptide (OATP2). Identification of a liver-specific human organic anion transporting polypeptide and identification of rat and human hydroxymethylglutaryl-CoA reductase inhibitor transporters. J Biol Chem 1999; 274(52):37161-8.
16 Abe T, Kakyo M, Tokui T, et al. Identification of a novel gene family encoding human liver-specific organic anion transporter LST-1. J Biol Chem 1999; 274(24):17159-63.
17 Tokui T, Nakai D, Nakagomi R, Yawo H, Abe T, Sugiyama Y. Pravastatin, an HMG-CoA reductase inhibitor, is transported by rat organic anion transporting polypeptide, oatp2. Pharm Res 1999; 16(6):904-8.
18 Konig J, Cui Y, Nies A T, Keppler D. A novel human organic anion transporting polypeptide localized to the basolateral hepatocyte membrane. Am J Physiol Gastrointest Liver Physiol 2000; 278(1):G156-64.
19 Nakai D, Nakagomi R, Furuta Y, et al. Human liver-specific organic anion transporter, LST-1, mediates uptake of pravastatin by human hepatocytes. J Pharmacol Exp Ther 2001; 297(3):861-7.
20 Cui Y. Konig J, Leier I, Buchholz U, Keppler D. Hepatic uptake of bilirubin and its conjugates by the human organic anion transporter SLC21A6. J Biol Chem: 2001; 276(13):9626-30.
21 Tirona R G, Leake B F, Merino G, Kim R B. Polymorphisms in OATP-C: identification of multiple allelic variants associated with altered transport activity among European- and African-Americans. J Biol Chem 2001; 276(38):35669-75.
22 Tirona R G, Kim R B. Pharmacogenomics of organic anion-transporting polypeptides (OATP). Adv Dig Deliv Rev 2002; 54(10):1343-52.
23 Nozawa T, Nakajima M, Tamai. I, et. al. Genetic polymorphisms of human organic anion transporters OATP-C (SLC21A6) and OATP-B (SLC21A9): allele frequencies in the Japanese population and functional analysis. J Pharmacol Exp Ther 2002; 302(2):804-13.
24 Michalski C, Cui Y, Nies. A T, et al. A naturally occurring mutation in the SLC21A6 gene causing impaired membrane localization of the hepatocyte uptake transporter. J Biol Chem 2002; 277(45):43058-63.
Nishizato Y, Ieiri I, Suzuki H, et al. Polymorphisms of OATP-C (SLC21A6) and OAT3 (SLC22A8) genes: consequences for pravastatin pharmacokinetics. Clin Pharmacol Ther 2003; 73(6) 554-65.
26 Bruckert E, Lievre M, Giral P, et al. Short-term efficacy and safety of extended-release fluvastatin in a large cohort of elderly patients. Am J Geriatr Cardiol 2003; 12(4):225-31.
27 de Kok J B, Wiegerinck E T, Giesendorf B A, Swinikels D W. Rapid genotyping of single nucleotide polymorphisms using novel minor groove binding DNA oligonucleotides (MGB probes). Hum Mutat 2002; 19(5):5549.
28 McTaggart F, Buckett L, Davidson. R, et al. Preclinical and clinical pharmacology of Rosuvastatin, a new 3-hydroxy-3-methylglutaryl coenzyme A reductase inhibitor. Am J Cardiol 2001; 87(5A):28B-32B.
29 Shitara Y, Itoh T, Sato H, Li A P, Sugiyama Y. Inhibition of transporter-mediated hepatic uptake as a mechanism for drug-drug interaction between cerivastatin and cyclosporin A. J Pharmacol Exp Ther 2003; 304(2):610-6.
30 Gould A L, Rossouw J E, Santanello N C, Heyse J F, Furberg C D. Cholesterol reduction yields clinical benefit: impact of statin trials. Circulation 1998; 97(10):946-52.

1. An ex vivo method for determining variable response to statin therapy in patients afflicted with or susceptible to develop cardiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level, comprising detecting the presence or absence of the Pro155Thr (C463A) variant in the Organic Anion Transporting Polypeptide-C(OATP-C) gene, wherein the presence of said variant is indicative of superior response to statin therapy (high responder versus low responder).

2. The method according to claim 1, wherein said statin includes atorvastatin, fluvastatin, lovastatin, pravastatin, rosuvastatin, simvastatin.

3. The method according to claim 1 comprising:
(a) obtaining a nucleic acid sample from the patient
(b) detecting the presence or absence of the C463A variant of OATP-C gene, in said acid nucleic sample
wherein the presence of said variant is indicative of superior response to statin therapy.

4. The method according to claim 3, wherein it comprises the use of primers and probes designed to specifically detect the C463A variant within the Organic Anion Transporting Polypeptide-C(OATP-C) gene sequence of SEQ ID No 1.

5. The method according to claim 4, wherein said specific probes are selected from a sequence from 10 to 35 nucleotide long surrounding and comprising the nucleotide at position 463 (numbered from the translation initiation start), preferably a 15 to 20 nucleotide long fragment of taatcaaatt ttatcactca atagagcatc a(c/a)⁴⁶³ctgagata gtgggaaaag gttgtttaaa (SEQ ID No 3) and comprising the nucleotide c or a at position 463.

6. The method according to claim 4, wherein probes are labelled with fluorescent labels.

7. The method according to claim 4, wherein primers for PCR amplification are:
Forward primer of
SEQ ID No 4
5′ AATTCAACATCGACCTTATCCACTTGT3′
Reverse primer
SEQ ID No 5
5′ACTGTCAATATTAATTCTTACCTTTTCCCACTATC 3′
and
wherein probes are
MGB probe wildtype
SEQ ID No 6
5′ VIC-CTCAATAGAGCATCACCTG-NFQ-MGB 3′
MGB probe mutant
SEQ ID No 7
5′ FAM-CAATAGAGCATCAACTG-NFQ-MGB 3′;
and wherein VIC and FAM code for the reporter fluorophores, NFQ corresponds to a non-fluorescent quencher and MGB represents the minor groove binding group.

8. The method according to claim 7 comprising a) nucleic acid extraction and purification, PCR amplification, b) hybridization under stringents conditions with two probes consisting of a 15 to 20 nucleotide long fragment of taatcaaatt ttatcactca atagagcatc a(c/a)⁴⁶³ ctgagata gtgggaaaag gttgtttaaa (SEQ ID No 3) and comprising the nucleotide c or a at position 463, preferably SEQ ID No 6 and 7 and c) signal detection.

9. The method according to claim 1 comprising:
(a) obtaining sample from the patient
(b) detecting the presence or absence of the Pro155Thr variant of OATP-C protein, in said nucleic acid sample
wherein the presence of said variant is indicative of superior response to statin therapy.

10. A kit for determining variable response to statin in patients afflicted with or susceptible to develop cardiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level, comprising primers and probes as defined in claim 5 for detecting the presence or absence of the C463A variant in the Organic Anion Transporting Polypeptide-C(OATP-C) gene.

11. The kit according to claim 10 further comprising a thermoresistant polymerase for PCR amplification and solutions for amplification and hybridization steps.

12. A method for treating and/or preventing or delaying the onset of cardiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level; comprising administering a decreased or increased daily dose of statin in homozygous Pro/Pro155 genotyped patients (low responders) and to homozygous Thr/Thr155 and heterozygous Pro/Thr155 genotyped patients (high responders) in the Organic Anion Transporting Polypeptide-C(OATP-C) gene, said increase or decrease being in the range of 10 to 100%, for example from 25% to 50%, 25% to 40%, 15% to 30% or 15% to 20% or 10% to 20% compared to the following equipotent doses:

| | | Tablet sizes | Initial dose | Equipotent dose¹⁹ |
|---|---|---|---|---|
| Generic Name | Trade Name | (mg) | (mg) | (mg) |
| Atorvastatin | Lipitor | 10, 20, 40, 80 | 10, 20, 40 | 10 |
| Fluvastatin | Lescol | 20, 40 | 20 or 40 in | 80* |
| | | | evening | |
| Fluvastatin | Lescol XL | 80 | 80 in evening | 80* |
| extended release | | | | |
| Lovastatin | Generic | 10, 20, 40 | 20 in evening | 60* |
| Lovastatin | Mevacor | 10, 20, 40 | 20 in evening | 60* |
| Lovastatin | Altocor | 10, 20, 40, 60 | 20, 40, or 60 | 40*²¹ |
| extended release | | | at bedtime | |
| Pravastatin | Pravachol | 10, 20, 40, 80 | 40 | 60* |
| Simvastatin | Zocor | 5, 10, 20, 40, 80 | 20 (40 in | 20-30* |
| | | | diabetes) | |

Taken from Buse J., Clinical Diabetes Vol. 21, No 4, 2003

13. A method for treating and/or preventing or delaying the onset of atherogenic dyslipidemias, type 2 diabetes, metabolic syndrome), stroke, peripheral vascular disease, the dyslipidemia associated with renal and neurodegenerative diseases and atherosclerosis with or without low plasma HDL-C levels, comprising administering a decreased or increased daily dose of statin in homozygous Pro/Pro155 genotyped patients (low responders) and to homozygous Thr/Thr155 and heterozygous Pro/Thr155 genotyped patients (high responders) in the Organic Anion Transporting Polypeptide-C(OATP-C) gene, said increase or decrease being in the range of 10 to 100%, for example from 25% to 50%, 25% to 40%, 15% to 30% or 15% to 20% or 10% to 20% compared to the equipotent doses defined in claim 12.

14. A method for treating and/or preventing or delaying the onset of cadiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level, atherogenic dyslipidemias, type 2 diabetes, metabolic syndrome), stroke, peripheral vascular disease, the dyslipidemia associated with renal and neurodegenerative diseases and atherosclerosis with or without low plasma HDL-C levels; comprising a frequency of statin administration to homozygous Pro/Pro155 genotyped patients (low responders) and to homozygous Thr/Thr155 and heterozygous Pro/Thr155 genotyped patients (high responders) in the Organic Anion Transporting Polypeptide-C(OATP-C) gene, said increase or decrease being in the range of 10 to 100%, for example from 25% to 50%, 25% to 40%, 15% to 30% or 15% to 20% or 10% to 20% compared to frequency of treatment regimen.

15. A method for combined tailored treatment and/or prevention of cardiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level comprising administering a statin and a PPARalpha agonist, such as a fibrat, according to the Pro155Thr variant in the Organic Anion Transporting Polypeptide-C(OATP-C) gene, especially to the population of low responder patients (Pro/Pro 155 genotyped patients).

16. A method for combined tailored treatment and/or prevention of cardiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level, atherogenic dyslipidemias, type 2 diabetes, metabolic syndrome), stroke, peripheral vascular disease, the dyslipidemia associated with renal and neurodegenerative diseases and atherosclerosis with or without low plasma HDL-C levels; wherein lower doses of statin are administered combined with fibrate or lower fibrate doses are administered combined with statin or both lower fibrate and lower statin doses are associated according to the Pro155Thr variant in the Organic Anion Transporting Polypeptide-C(OATP-C) gene, especially to the population of high responder patients (Thr/Thr155 and Pro/Thr155).

17. A method for combined therapy and prevention for high stastin responder patients (Thr/Thr155 and Pro/Thr155 in the Organic Anion Transporting Polypeptide-C(OATP-C) gene) and for low statin responder patients (Pro/Pro155) comprising administering
Statin+nicotinic acid (Niacin) or derivatives (i.e Niaspan®) or other nicotinic acid receptor agonists
Statin+bile binding Resin (i.e cholestyramine, Questran®; Colesevelam, Colestipol, Welchol)
Statin+CETP inhibitors (i.e Torcetrapib®)
Statin+cholesterol adsorption inhibitors (ex Ezitimibe, Ezetrol®)
as well as any combination thereof (i.e statin+niacin+resin).

18. The use of Fluvastatin for preparing a medicament suitable for administration of 80 mg/day or more, for example from 85 to 120 mg/day, 90 to 95 mg/day or 90 to 110 mg/day, for example 85, 90, 95, 100, 105, 110, 115, 120 mg/day to the homozygous Pro/Pro155 genotyped patients in the Organic Anion Transporting Polypeptide-C(OATP-C) gene for treating and/or preventing or delaying the onset of cadiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level.

19. The use of Fluvastatin for preparing a medicament suitable for administration of less than 80 mg/day, for example from 75 to 20 mg/day, 70 to 50 mg/day or 60 to 50 mg/day, for example 75, 70, 65, 60, 50, 45, or 40 mg/day to the Thr/Thr155 and Pro/Thr155 genotyped patients in the Organic Anion Transporting Polypeptide-C(OATP-C) gene for treating and/or preventing or delaying the onset of cadiovascular diseases such as coronary artery diseases, ischaemic heart disease and myocardial infarct, hypercholesterolemia, Diabetes Mellitus, atherosclerosis and/or any diseases or metabolic disorders involving high baseline plasma lipid level such as high LDL-C level.

20. The use according to claim 18 for preparing a medicament for treating and/or preventing or delaying the onset of atherogenic dyslipidemias, type 2 diabetes, metabolic syndrome), stroke, peripheral vascular disease, the dyslipidemia associated with renal and neurodegenerative diseases and atherosclerosis with or without low plasma HDL-C levels, as well as renal transplantation patients.


SEQUENCE LISTING (7 sequences)

SEQ ID NO: 1 - DNA, 2452 bp, Homo sapiens
  OATP-C showing the C463A variant being numbered from the start codon and corresponding to NCBI AB026257
gtggacttgttgcagttgctgtaggattctaaatccaggtgattgtttcaaactgagcatcaacaacaaaaacatttgtatgatatctatatttcaatcatggaccaaaatcaacatttgaataaaacagcagaggcacaaccttcagagaataagaaaacaagatactgcaatggattgaagatgttcttggcagctctgtcactcagctttattgctaagacactaggtgcaattattatgaaaagttccatcattcatatagaacggagatttgagatatcctcttctcttgttggttttattgacggaagctttgaaattggaaatttgcttgtgattgtatttgtgagttactttggatccaaactacatagaccaaagttaattggaatcggttgtttcattatgggaattggaggtgttttgactgctttgccacatttcttcatgggatattacaggtattctaaagaaactaatatcaattcatcagaaaattcaacatcgaccttatccacttgtttaattaatcaaattttatcactcaatagagcatcamctgagatagtgggaaaaggttgtttaaaggaatctgggtcatacatgtggatatatgtgttcatgggtaatatgcttcgtggaataggggagactcccatagtaccactggggctttcttacattgatgatttcgctaaagaaggacattcttctttgtatttaggtatattgaatgcaatagcaatgattggtccaatcattggctttaccctgggatctctgttttctaaaatgtacgtggatattggatatgtagatctaagcactatcaggataactcctactgattctcgatgggttggagcttggtggcttaatttccttgtgtctggactattctccattatttcttccataccattctttttcttgccccaaactccaaataaaccacaaaaagaaagaaaagcttcactgtctttgcatgtgctggaaacaaatgatgaaaaggatcaaacagctaatttgaccaatcaaggaaaaaatattaccaaaaatgtgactggttttttccagtcttttaaaagcatccttactaatcccctgtatgttatgtttgtgcttttgacgttgttacaagtaagcagctatattggtgcttttacttatgtcttcaaatacgtagagcaacagtatggtcagccttcatctaaggctaacatcttattgggagtcataaccatacctatttttgcaagtggaatgtttttaggaggatatatcattaaaaaattcaaactgaacaccgttggaattgccaaattctcatgttttactgctgtgatgtcattgtccttttacctattatattttttcatactctgtgaaaacaaatcagttgccggactaaccatgacctatgatggaaataatccagtgacatctcatagagatgtaccactttcttattgcaactcagactgcaattgtgatgaaagtcaatgggaaccagtctgtggaaacaatggaataacttacatctcaccctgtctagcaggttgcaaatcttcaagtggcaataaaaagcctatagtgttttacaactgcagttgtttggaagtaactggtctccagaacagaaattactcagcccatttgggtgaatgcccaagagatgatgcttgtacaaggaaattttacttttttgttgcaatacaagtcttgaatttatttttctctgcacttggaggcacctcacatgtcatgctgattgttaaaattgttcaacctgaattgaaatcacttgcactgggtttccactcaatggttatacgagcactaggaggaattctagctccaatatattttggggctctgattgatacaacgtgtataaagtggtccaccaacaactgtggcacacgtgggtcatgtaggacatataattccacatcattttcaagggtctacttgggcttgtcttcaatgttaagagtctcatcacttgttttatatattatattaatttatgccatgaagaaaaaatatcaagagaaagatatcaatgcatcagaaaatggaagtgtcatggatgaagcaaacttagaatccttaaataaaaataaacattttgtcccttctgctggggcagatagtgaaacacattgttaaggggagaaaaaaagccacttctgcttctgtgtttccaaacagcattgcattgattcagtaagatgttatttttgaggagttcctggtcctttcactaagaatttccacatcttttatggtggaagtataaataagcctatgaacttataataaaacaaactgtaggtagaaaaaatgagagtactcattgtacattatagctacatatttgtggttaaggttagactatatgatccatacaaattaaagtgagagacatggttactgtgtaataaaa

SEQ ID NO: 2 - protein, 691 aa, Homo sapiens
  OATP-C peptidic sequence showing the variant P/T at position 155
MDQNQHLNKTAEAQPSENKKTRYCNGLKMFLAALSLSFIAKTLGAIIMKSSIIHIERRFEISSSLVGFIDGSFEIGNLLVIVFVSYFGSKLHRPKLIGIGCFIMGIGGVLTALPHFFMGYYRYSKETNINSSENSTSTLSTCLINQILSLNRASXEIVGKGCLKESGSYMWIYVFMGNMLRGIGETPIVPLGLSYIDDFAKEGHSSLYLGILNAIAMIGPIIGFTLGSLFSKMYVDIGYVDLSTIRITPTDSRWVGAWWLNFLVSGLFSIISSIPFFFLPQTPNKPQKERKASLSLHVLETNDEKDQTANLTNQGKNITKNVTGFFQSFKSILTNPLYVMFVLLTLLQVSSYIGAFTYVFKYVEQQYGQPSSKANILLGVITIPIFASGMFLGGYIIKKFKLNTVGIAKFSCFTAVMSLSFYLLYFFILCENKSVAGLTMTYDGNNPVTSHRDVPLSYCNSDCNCDESQWEPVCGNNGITYISPCLAGCKSSSGNKKPIVFYNCSCLEVTGLQNRNYSAHLGECPRDDACTRKFYFFVAIQVLNLFFSALGGTSHVMLIVKIVQPELKSLALGFHSMVIRALGGILAPIYFGALIDTTCIKWSTNNCGTRGSCRTYNSTSFSRVYLGLSSMLRVSSLVLYIILIYAMKKKYQEKDINASENGSVMDEANLESLNKNKHFVPSAGADSETHC

SEQ ID NO: 3 - DNA, 60 bp, artificial sequence
  segment of OATP-C coding sequence displaying the variant c/a at position 463
taatcaaattttatcactcaatagagcatcamctgagatagtgggaaaaggttgtttaaa

SEQ ID NO: 4 - DNA, 27 bp, artificial sequence
  forward primer
aattcaacatcgaccttatccacttgt

SEQ ID NO: 5 - DNA, 35 bp, artificial sequence
  reverse primer
actgtcaatattaattcttaccttttcccactatc

SEQ ID NO: 6 - DNA, 19 bp, artificial sequence
  MGB probe wildtype
ctcaatagagcatcacctg

SEQ ID NO: 7 - DNA, 17 bp, artificial sequence
  MGB probe mutant
caatagagcatcaactg